(19) United States
(12) Patent Application Publication
(10) Pub. No.: US 2010/0021405 A1
(43) Pub. Date: Jan. 28, 2010

(54) EXTERNAL PREPARATION FOR SKIN

(76) Inventors: Masamichi Abe, Osaka-fu (JP); Ayako Harada, Osaka-fu (JP); Yoichi Honma, Osaka-fu (JP)

Correspondence Address: BIRCH STEWART KOLASCH & BIRCH, PO BOX 747, FALLS CHURCH, VA, 22040-0747, US

(21) Appl. No.: 12/445,177

(22) PCT Filed: Oct. 11, 2007
(86) PCT No.: PCT/JP2007/069860

§ 371 Date: Apr. 10, 2009

(30) Foreign Application Priority Data
Oct. 12, 2006 (JP) 2006-279252

(51) Int. Cl. A61K 8/18; A61K 31/66; A61Q 19/02

(52) U.S. Cl. 424/62; 514/75

(57) ABSTRACT

The present invention provides an external preparation for skin comprising a phospholipid having an iodine value of 80 to 110, ethanol in an amount of 55 to 83 wt % and water in an amount of 15 to 43 wt %, which is improved in it's preparation stability by suppressing an increase in an acid value of phospholipid. The present invention also provides an external preparation for skin which is improved in percutaneous absorption of medically effective ingredient(s). The present invention provides further a method for suppressing an increase in an acid value of phospholipid by comprising phospholipid having high iodine value and high concentration of ethanol and water, as well as a method for improving percutaneous absorption of medically effective ingredient(s) in external preparation for skin.

### TECHNICAL FIELD

This invention relates to an external preparation for skin in which preparation stability is improved by suppressing an increase in an acid value of phospholipid. This invention also relates to an external preparation for skin in which percutaneous absorbability of medically effective ingredient(s) is improved remarkably.

### BACKGROUND ART

Phospholipids naturally occur in animals and plants, such as soybeans, egg yolks, etc., and most phospholipids are highly safe ingredients that can be used in food, and are known to have surface-active effects and moisturizing properties, and are also known to promote percutaneous absorption of the medically effective ingredient(s) when phospholipids are contained in the external preparation for skin. However phospholipids changes in quality by heat or light because of intrinsic property of lipid, which thus results in a release of fatty acid, namely rancidity, and an increase of an acid value. According to Japanese Standards of Cosmetic Ingredients, the acid value of soybean phospholipid is defined as 40 or less, and thus it is required for stabilizing a preparation by suppressing an increase in the acid value of phospholipid in the preparation.

Also the external preparation for use on the skin or mucosa is available in various forms such as patches, ointments, creams, lotions, solid preparations, etc. But it is not easy for a useful ingredient incorporated in the external preparation for skin to permeate efficiently through the skin since permeation is inhibited by the stratum corneum, which prevents external foreign substances from entering. Thus various studies have been made to promote percutaneous absorption, and there have been reports on external preparations with being improved in percutaneous absorption, such as a composition comprising phospholipid, ethanol in an amount of 50% or less percent by weight (hereinafter abbreviated as wt %) and water (see Japanese Unexamined Patent Application Publication No. 2004-536089) and a composition for promotion of absorption comprising a phospholipid and a specific polyhydric alcohol (see Japanese Unexamined Patent Application Publication No. 1998-194994), etc.

### DISCLOSURE OF INVENTION

### Problems to be Solved by the Invention

An object of the present invention is to provide an external preparation for skin in which preparation stability is improved in by suppressing an increase in an acid value of phospholipid. Another object of the present invention is to provide an external preparation for skin in which percutaneous absorbability of medically effective ingredient(s) is improved in remarkably.

### Means for Solving Problem

The present inventors have intensively studied in order to achieve the above-mentioned objects, and have found that an increase in an acid value of phospholipid can be suppressed by adding ethanol in an amount of 55 to 83 wt % and water in an amount of 15 to 43 wt % to a composition comprising a phospholipid having an iodine value of 80 to 110, also that percutaneous absorbability of medically effective ingredient(s) is improved remarkably by incorporating a phospholipid having an iodine value of 80 to 110, ethanol in an amount of 55 to 83 wt % and water in an amount of 15 to 43 wt % into medically effective ingredient(s), and have accomplished the present invention.

That is, the present invention provides an external preparation for skin as set forth in the following embodiments [1] to [5]:
[1] An external preparation for skin comprising a phospholipid having an iodine value of 80 to 110, ethanol in an amount of 55 to 83 wt % and water in an amount of 15 to 43 wt %.
[2] The external preparation for skin as set forth in [1], further comprising one or two or more kind(s) of ingredient(s) selected from the group consisting of glycol, glycol ether, glycerol and diglycerol in an amount of 5 to 29 wt %.
[3] The external preparation for skin as set forth in either [1] or [2], further comprising medically effective ingredient(s).
[4] The external preparation for skin as set forth in [3], wherein the medically effective ingredient(s) is one or two or more kind(s) of substance(s) selected from the group consisting of vitamin A compounds, vitamin C compounds, skin-whitening agents, anti-wrinkle agents, anti-inflammatory analgesics, antifungals, steroids, hair restorers, slimming agents and antipruritics.
[5] The external preparation for skin as set forth in [3], wherein the medically effective ingredient(s) is one or two or more kind(s) of substance(s) selected from the group consisting of anti-inflammatory analgesics, antifungals, steroids, hair restorers and antipruritics.

The present invention also provides a method for suppressing an increase in an acid value of phospholipid as set forth in the following embodiments [6] to [7]:
[6] A method for suppressing an increase in an acid value of phospholipid characterized by coexistence of ethanol in an amount of 55 to 83 wt % and water in an amount of 15 to 43 wt % with a phospholipid having an iodine value of 80 to 110.
[7] The method as set forth in [6], characterized by further coexistence of one or two or more kind(s) of ingredient(s) selected from the group consisting of glycol, glycol ether, glycerol and diglycerol in an amount of 5 to 29 wt %.

The present invention further provides a method for improving in percutaneous absorbability of medically effective ingredient(s) of an external preparation for skin as set forth in the following embodiments [8] to [9]:
[8] A method for improving in percutaneous absorbability of medically effective ingredient(s) of an external preparation for skin, characterized by coexistence of a phospholipid having an iodine value of 80 to 110, ethanol in an amount of 55 to 83 wt % and water in an amount of 15 to 43 wt % with the medically effective ingredient(s).
[9] The method as set forth in [8], characterized by further coexistence of one or two or more kind(s) of ingredient(s) selected from the group consisting of glycol, glycol ether, glycerol and diglycerol in an amount of 5 to 29 wt %.

### Effect of the Invention

According to the present invention, an increase in an acid value of phospholipid incorporated into an external preparation for skin can be suppressed by containing phospholipid having high iodine value and specific amounts of ethanol and water, which thus can be expected to improve in a preparation stability of an external preparation for skin. Further according to the present invention, percutaneous absorbability of medically effective ingredient(s) incorporated into an external preparation for skin can be promoted by containing phospholipid and specific amounts of ethanol and water, which thus can be expected to achieve efficient permeation of medically effective ingredient(s) through the skin.

### BEST MODES FOR CARRYING OUT THE INVENTION

The present invention is explained in more detail below.

The following are definitions of terms used in this description and claims.

The external preparation for skin of the present invention is characterized by comprising a phospholipid having an iodine value of 80 to 110, ethanol in an amount of 55 to 83 wt % and water in an amount of 15 to 43 wt %. The method for suppressing an increase in an acid value of a phospholipid according to the present invention is characterized by incorporating ethanol and water to the phospholipid.

Phospholipids for use in the present invention are one of components of cell, are high-biocompatible, and are useful as ingredient of external preparation for skin.

The phospholipids include those having a wide range of iodine value and the phospholipid for use in the present invention is those having high iodine value.

Specific examples of the phospholipid for use in the present invention include among glycerophospholipids and sphingophospholipids, and the like, those having an iodine value of 80 to 110.

Glycerophospholipids are materials with glycerophosphate skeletons, which contain fatty acid ester, a long-chain alkyl ether and vinyl ether, and the like, as lipophilic moiety. Specific examples include phosphatidylcholine, phosphatidylethanolamine, phosphatidylserine, phosphatidylinositol, phosphatidylinositol polyphosphate, phosphatidylglycerol, diphosphatidylglycerol (cardiolipin), phosphatidic acid, lysophosphatidylcholine, lysophosphatidylethanolamine, lysophosphatidylserine, lysophosphatidylinositol, lysophosphatidylglycerol and lysophosphatidic acid, and the like.

Sphingophospholipids are materials containing a long-chain base or a long-chain fatty acid such as sphingosine, phytosphingosine, etc., and phosphoric acid or phosphonic acid, and include specifically those ceramide-1-phosphate derivatives such as sphingomyelin, etc., and ceramide-1-phosphonate derivatives such as ceramide aminoethylphosphonate, etc., and the like.

Among these phospholipids, glycerophospholipids are preferable, with phosphatidylcholine, phosphatidylethanolamine and phosphatidylglycerol being particularly preferable.

In addition, the phospholipids for use in the present invention may be any of natural phospholipids that have been extracted and purified from animals or plants, and chemically synthesized phospholipids. Commercially available phospholipids can also be used. The natural phospholipids are preferably lecithins, which are extracted and purified from soybeans, egg yolks, and the like.

The incorporated amount of phospholipid used in the present invention is not limited otherwise as long as the effects of the present invention are not impaired, but is usually 0.01 to 15 wt %, preferably 0.05 to 10 wt %, particularly preferably 0.1 to 8 wt %, based on the total amount of external preparation for skin.

The external preparation for skin of the present invention comprises ethanol and water, in which the incorporated amounts thereof are described as follows. That is, the incorporated amount of ethanol is usually 55 to 83 wt %, preferably 55 to 80 wt %, more preferably 55 to 75 wt %, further more preferably 60 to 75 wt %, based on the total amount of external preparation for skin. The incorporated amount of water is usually 15 to 43 wt %, preferably 20 to 40 wt %, more preferably 20 to 35 wt %, based on the total amount of external preparation for skin.

Further the external preparation for skin of the present invention can be prepared by optionally incorporating ingredient(s) selected from the group consisting of glycol, glycol ether, glycerol and diglycerol alone or in a combination of two or more kinds thereof in appropriate amounts to the above external preparation for skin in order to improve in preparation stability by suppressing the increase in an acid value of the phospholipid.

The glycol for use in the present invention is a diol as liquid at 25° C., which is used as an ingredient for external preparation for skin in the fields of pharmaceuticals, quasi drugs or cosmetics, and specifically includes a diol represented by a general formula of CₙH₂ₙ(OH)₂ or condensate products of one or two or more kind(s) of the above diol(s), and the like. Specific examples include ethylene glycol, propylene glycol, trimethylene glycol, 1,2-butylene glycol, 1,3-butylene glycol, 2,3-butylene glycol, isoprene glycol, 1,2-pentylene glycol, 1,2-hexylene glycol and octylene glycol, and the like; as the condensate products, diethylene glycol, triethylene glycol, tetraethylene glycol, dipropylene glycol and tripropylene glycol and the like. Preferred examples are propylene glycol, 1,3-butylene glycol and dipropylene glycol, and the like.

The glycol ether for use in the present invention is a compound in which one or both of the hydroxy group(s) of the above glycol is/are etherified, and is not limited otherwise as long as they are those used generally as an ingredient for external preparation for skin in the fields of pharmaceuticals, quasi drugs or cosmetics.

Specific examples of glycol ether include ethylene glycol monomethyl ether, ethylene glycol monoethyl ether, ethylene glycol monopropyl ether, diethylene glycol monomethyl ether, diethylene glycol monoethyl ether (ethoxydiglycol), diethylene glycol monopropyl ether, diethylene glycol monobutyl ether, propylene glycol monoethyl ether, propylene glycol monopropyl ether, dipropylene glycol monoethyl ether and dipropylene glycol monopropyl ether, and the like, with diethylene glycol monoethyl ether, diethylene glycol monobutyl ether being particularly preferable.

Also glycerol and diglycerol for use in the present invention are well-known compounds that are used frequently for external preparation for skin, and the like.

These glycol, glycol ether, glycerol and diglycerol can be used alone or in a combination of two or more kinds thereof, and the total amount of glycol, glycol ether, glycerol and diglycerol is 1 to 29 wt %, preferably 1 to 20 wt %, particularly preferably 1 to 10 wt %, based on the total amount of the external preparation for skin, but is not limited otherwise as long as the effects of the present invention are not impaired.

Also in the external preparation for skin of the present invention, a ratio of the total amount of glycol, glycol ether, glycerol and diglycerol to an amount of phospholipid is usually 1 to 300 part by weight, preferably 2 to 100 part by weight, particularly preferably 3 to 50 part by weight per part by weight of phospholipid, but is not limited otherwise as long as the effects of the present invention are not impaired.

For example, the external preparation for skin of the present invention can be prepared by dissolving a phospholipid in ethanol, followed by mixing the resulting mixture with purified water that is warmed separately. The external preparation for skin of the present invention can also be prepared by dissolving a phospholipid into a mixed solution of ethanol and one or two or more kind(s) of ingredient(s) selected from the group consisting of glycol, glycol ether, glycerol and diglycerol, followed by mixing the resulting mixture with purified water that is warmed separately.

The following each various medically effective ingredient(s) can be incorporated into the external preparation for skin of the present invention.

The medically effective ingredient(s) for use in the present invention is not limited otherwise as long as it is an ingredient useful for the skin such as a pharmacological active ingredient or a bioactive ingredient, etc., and include specifically vitamin compounds (such as vitamin A compounds, provitamin A compounds, vitamin E compounds, vitamin B2 compounds, nicotinic acid compounds, vitamin C compounds (water-soluble or water-insoluble), vitamin D compounds, vitamin K compounds, vitamin B1 compounds, vitamin B6 compounds, vitamin B12 compounds, folic acid compounds, pantothenic acid compounds, biotin compounds, vitamin-like active factors, etc.), skin-whitening agents, anti-wrinkle agents, anti-inflammatory analgesics, antifungals, steroids, hair restorers, slimming agents, local anesthetics, antipruritics, antimicrobials, antivirals, keratin softeners, moisturizers, astringents, antioxidants and hair growth inhibitors, and the like, with vitamin A compounds, vitamin C compounds (water-soluble or water-insoluble), skin-whitening agents, anti-wrinkle agents, anti-inflammatory analgesics, antifungals, steroids, hair restorers, slimming agents and antipruritics being preferable, vitamin A compounds, water-soluble vitamin C compounds, anti-wrinkle agents, anti-inflammatory analgesics, antifungals, steroids, hair restorers, slimming agents and antipruritics being more preferable, and anti-inflammatory analgesics, antifungals, steroids, hair restorers and antipruritics being particularly preferable.

These ingredients can be used alone or in a combination of two or more kinds thereof.

Specific examples of the medically effective ingredients are shown below.

Examples of the vitamin compounds include retinol derivatives such as retinol, retinol acetate, etc.; vitamin A compounds such as retinal, retinoic acid, methyl retinoate, ethyl retinoate, retinol retinoate, vitamin A oil, vitamin A fatty acid esters, d-δ-tocopheryl retinoate, α-tocopheryl retinoate, β-tocopheryl retinoate, etc.; provitamin A compounds such as β-carotene, α-carotene, γ-carotene, δ-carotene, lycopene, zeaxanthin, cryptoxanthin, echinenone, etc.; vitamin E compounds such as dl-α-tocopherol succinate, dl-α-tocopherol calcium succinate, δ-tocopherol, etc.; vitamin B2 compounds such as riboflavin, flavin mononucleotide, flavin adenine dinucleotide, riboflavin butyrate, riboflavin tetrabutylate, sodium riboflavin-5′-phosphate, riboflavin tetranicotinate, etc.; nicotinic acid compounds such as methyl nicotinate, nicotinic acid, nicotinamide, etc.; vitamin C compounds such as ascorbyl stearate, L-ascorbyl dipalmitate, ascorbyl tetraisopalmitate (ascorbyl tetra-2-hexyl decanoate), ascorbic acid, sodium ascorbate, dehydroascorbic acid, sodium ascorbyl phosphate, magnesium ascorbyl phosphate, ascorbyl glucoside, etc.; vitamin D compounds such as methylhesperidin, ergocalciferol, cholecalciferol, etc.; vitamin K compounds such as phylloquinone, farnoquinone, etc.; vitamin B1 compounds such as dibenzoyl thiamine, dibenzoyl thiamine hydrochloride, thiamine hydrochloride, thiamine cetyl hydrochloride, thiamine thiocyanate, thiamine lauryl hydrochloride, thiamine nitrate, thiamine monophosphate, lysine salt of thiamine, thiamine triphosphate, phosphoric acid salt of thiamine monophosphate, thiamine monophosphate, thiamine diphosphate, thiamine diphosphate hydrochloride, thiamine triphosphate, monophosphoric acid salt of thiamine triphosphate, etc.; vitamin B6 compounds such as pyridoxine hydrochloride, pyridoxine acetate, pyridoxal hydrochloride, pyridoxal 5′-phosphate, pyridoxamine hydrochloride, etc.; vitamin B12 compounds such as cyanocobalamin, hydroxocobalamin, deoxyadenosylcobalamin, etc.; folic acid compounds such as folic acid, pteroylglutamic acid, etc.; pantothenic acid compounds such as pantothenic acid, calcium pantothenate, pantothenyl alcohol (panthenol), D-pantesin, D-pantethine, coenzyme A, pantothenyl ethyl ether, etc.; biotin compounds such as biotin, bioticin, etc.; and vitamin-like active factors such as carnitine, ferulic acid, α-lipoic acid, orotic acid, γ-oryzanol, etc., and the like.

Among them, vitamin A compounds such as d-δ-tocopheryl retinoate, etc., vitamin C compouds such as ascorbyl tetraisopalmitate, ascorbic acid, ascorbyl glucoside, etc., and vitamin E compounds such as dl-α-tocopherol succinate, dl-α-tocopherol calcium succinate, δ-tocopherol, etc., are preferable, and vitamin A compounds such as d-δ-tocopheryl retinoate, etc., water-soluble vitamin C compounds such as ascorbic acid, ascorbyl glucoside, etc., and vitamin E compounds such as δ-tocopherol, etc., are particularly pereferable.

The incorporated amount of vitamin compounds used in the present invention is not limited otherwise, and can be suitably selected in view of a feeling on the skin and a pharmacological or a physiological effect. The incorporated amount of vitamin compounds is usually 0.1 to 29 wt %, preferably 0.5 to 25 wt %, particularly preferably 1 to 20 wt %, based on the total amount of the external preparation for skin.

Examples of skin-whitening agents include placentas, arbutin, cysteine, ellagic acid, kojic acid, phytic acid, rucinol, hydroquinone, orizanol; ingredients, extracts, and essential oils derived from plants such as iris, almond, aloe, ginkgo, oolong tea, rose fruit, scutellaria root, Coptis Rhizome, St. John's wort (Hypericum erectum Thunb), dead nettle, seaweed, pueraria root, chamomile, licorice, gardenia, Sophorae Radix, wheat, rice, rice germ, rice bran, perilla, peony, Cnidium Rhizome, mulberry bark, soybeans, tea, terminalia, Japanese angelica, Calendula officinalis, hamamelis, safflower, moutan bark, coix seeds, Japanese angelica, Celtis sinensis, persimmon (Diospyros kaki), clove, etc., and the like, with among them, arbutin, cysteine and terminalia extracts being preferable.

The incorporated amount of skin-whitening agents used in the present invention is not limited otherwise, and can be suitably selected in view of a feeling on the skin and a pharmacological or a physiological effect. The incorporated amount of skin-whitening agents is usually 0.1 to 29 wt %, preferably 0.5 to 25 wt %, particularly preferably 1 to 20 wt %, based on the total amount of the external preparation for skin.

Examples of the anti-wrinkle agents include coenzymes Q10, kinetin, glycolic acid, argireline, acylated glucosamine, collagens, hyaluronic acid, aloe extracts, seaweed extracts, horse chestnut extracts, rosemary extracts, cornflower extracts, and the like, with among them, coenzymes Q10, kinetin being preferable.

The incorporated amount of anti-wrinkle agents used in the present invention is not limited otherwise, and can be suitably selected in view of a feeling on the skin and a pharmacological or physiological effect. The incorporated amount of anti-wrinkle agents is usually 0.1 to 29 wt %, preferably 0.5 to 25 wt %, particularly preferably 1 to 20 wt %, based on the total amount of the external preparation for skin.

Examples of the anti-inflammatory analgesics include indomethacin, felbinac, methyl salicylate, glycol salicylate, allantoin or allantoin derivatives, ibuprofen, ibuprofen piconol, bufexamac, butyl flufenamate, bendazac, piroxicam, ketoprofen, and the like, with among them, indomethacin, felbinac and methyl salicylate being preferable.

The incorporated amount of anti-inflammatory analgesics used in the present invention is not limited otherwise, and can be suitably selected in view of a feeling on the skin and a pharmacological or physiological effect. The incorporated amount of anti-inflammatory analgesics is usually 0.1 to 29 wt %, preferably 0.5 to 25 wt %, more preferably 1 to 20 wt %, particularly preferably 1 to 15 wt %, based on the total amount of the external preparation for skin.

Examples of the antifungals include terbinafine, sulconazole, clotrimazole, isoconazole, cloconazole, miconazole, econazole, oxiconazole, butenafine, amorolfine, neticonazole and salts thereof (such as an acid-additional salt, preferably a salt with an inorganic acid such as nitrate, hydrochloride, etc.), bifonazole, tioconazole, ketoconazole, tolnaftate, tolciclate, liranaftate, ciclopirox olamine, exalamide, siccanin, undecylenic acid, zinc undecylenate and pyrrolnitrin, and the like, with among them, terbinafine hydrochloride, sulconazole nitrate, clotrimazole, isoconazole nitrate, cloconazol nitrate, miconazole nitrate, econazole nitrate, oxiconazole nitrate, bifonazole, tioconazole, ketoconazole, tolnaftate, tolciclate, liranaftate, ciclopirox olamine, exalamide, siccanin, undecylenic acid, zinc undecylenate, pyrrolnitrin, butenafine hydrochloride, amorolfine hydrochloride, neticonazole hydrochloride, and the like, being preferable, and terbinafine hydrochloride and sulconazole nitrate being particularly preferable.

The incorporated amount of antifungals used in the present invention is not limited otherwise, and can be suitably selected in view of a feeling on the skin and a pharmacological or physiological effect. The incorporated amount of antifungals is usually 0.1 to 29 wt %, preferably 0.1 to 25 wt %, more preferably 0.1 to 20 wt %, particularly preferably 0.1 to 10 wt %, based on the total amount of the external preparation for skin.

Examples of the steroids include dexamethasone, prednisolone, hydrocortisone, cortisone, betamethasone, clobetasone, clobetasol, diflorasone, diflucortolone, beclometasone, flumetasone and ester derivatives thereof (preferably ester derivatives with an acid such as acetic acid, propionic acid, butyric acid, valeric acid, pivalic acid, etc.), triamcinolone acetonide, fluocinolone acetonide, fluocinonide, amcinonide, halcinonide, difluprednate, and the like, with among them, dexamethasone valerate acetate, dexamethasone, dexamethasone propionate, dexamethasone acetate, dexamethasone valerate, prednisolone valerate acetate, hydrocortisone butyrate, hydrocortisone acetate, hydrocortisone, hydrocortisone butyrate propionate, cortisone acetate, prednisolone acetate, prednisolone, betamethasone, betamethasone valerate, betamethasone dipropionate, clobetasone butyrate, clobetasol propionate, diflorasone acetate, diflucortolone valerate, beclometasone propionate, flumetasone pivalate, triamcinolone acetonide, fluocinolone acetonide, fluocinonide, amcinonide, halcinonide, difluprednate, and the like, being preferable, and hydrocortisone acetate, hydrocortisone, hydrocortisone butyrate, prednisolone, prednisolone acetate, prednisolone valerate acetate, dexamethasone and dexamethasone acetate being more preferable.

The incorporated amount of steroids used in the present invention is not limited otherwise, and can be suitably selected in view of a feeling on the skin and a pharmacological or physiological effect. The incorporated amount of steroids is usually 0.01 to 1 wt %, preferably 0.01 to 0.7 wt %, particularly preferably 0.01 to 0.5 wt %, based on the total amount of the external preparation for skin.

Examples of the hair restorers include procyanidin, dipotassium glycyrrhizinate, carpronium chloride, cepharanthin, menthol, hinokitiol, L-hydroxyproline, acetyl hydroxyproline, fucoidan, capsicum tincture, cepharanthin, swertianine, flavonosteroids, minoxidil, FGF-10, vitamin E compounds and soybean protein hydrolysates, and the like. The hair restorers of the present invention include plant ingredients or plant extracts (essences) comprising the hair restorers illustrated above. The plant ingredients or plant extracts (essences) include Isodon japonicus Hara extracts (essences), Swertia japonica extracts (essences), Laminaria angustata extracts (essences), Gynostemma pentaphyllum extracts (essences), St. John's wort (Hypericum erectum Thunb) extracts (essences), gentian extracts (essences), sage extracts (essences), peppermint extracts (essences), hop extracts (essences), coix seed extracts (essences), persimmon leaf extracts (essences), Rehmanniae Radix extracts (essences), ginseng extracts (essences), Tilia miqueliana extracts (essences), moutan bark extracts (essences) and seaweed extracts, and the like. Preferred examples include procyanidin, Swertia japonica extracts (essences), Laminaria angustata extracts (essences), ginseng extracts (essences), menthol, dipotassium glycyrrhizinate, vitamin E compounds, soybean protein hydrolysates and seaweed extracts.

The incorporated amount of the hair restorers used in the present invention is not limited otherwise, and can be suitably selected in view of a feeling on the skin and a pharmacological or physiological effect. The incorporated amount of hair restorers is usually 0.05 to 29 wt %, preferably 0.05 to 25 wt %, more preferably 0.1 to 20 wt %, particularly preferably 0.1 to 10 wt %, based on the total amount of the external preparation for skin. Here when the hair restorers are incorporated as plant ingredients or plant extracts (essences), the incorporated amount is calculated based on an amount of the hair restorers contained in the plant ingredients or plant extracts (essences).

Examples of the slimming agents include xanthine compounds such as caffeine, aminophylline, theophylline, oxtriphylline, dyphylline, diisobutylaminobenzoyloxypropyl theophylline, theobromine, diprophylline, proxyphylline, pentoxifylline, etc.; and capsaicin, and the like, with among them, caffeine and capsaicin being preferable.

The incorporated amount of the slimming agents used in the present invention is not limited otherwise, and can be suitably selected in view of a feeling on the skin and a pharmacological or physiological effect. The incorporated amount of slimming agents is usually 0.00001 to 29 wt %, preferably 0.00001 to 25 wt %, particularly preferably 0.00001 to 20 wt %, based on the total amount of the external preparation for skin. Among others, when caffeine is incorporated as slimming agents, the incorporated amount is usually 0.1 to 10 wt %, preferably 0.5 to 5 wt %, based on the total amount of the external preparation for skin. Also when capsaicin is incorporated as slimming agents, the incorporated amount is usually 0.00001 to 0.01 wt %, preferably 0.0001 to 0.001 wt %, based on the total amount of the external preparation for skin.

Examples of the antipruritics include crotamiton, chlorpheniramine or a salt thereof (such as an acid-additional salt, preferably a salt with an organic acid such as maleic acid, etc.,), diphenhydramine or a salt thereof (such as an acid-additional salt, preferably salts with an inorganic acid such as hydrochloric acid, etc., or an organic acid such as salicylic acid, etc.,), salicylic acid, nonylic acid vanillylamide, mequitazine, camphor, thymol, eugenol, polyoxyethylene lauryl ether, comfrey extracts and perilla extracts, and the like, with among them, crotamiton, diphenhydramine or a salt thereof (such as diphenhydramine, diphenhydramine hydrochloride, etc.,) being preferable.

The incorporated amount of the antipruritics used in the present invention is not limited otherwise, and can be suitably selected in view of a feeling on the skin and a pharmacological or physiological effect. The incorporated amount of antipruritics is usually 0.001 to 20 wt %, preferably 0.01 to 15 wt %, particularly preferably 0.01 to 10 wt %, based on the total amount of the external preparation for skin.

Besides, specific examples of local anesthetics, antimicrobials, antivirals, keratin softeners, moisturizers, astringents, antioxidants and hair growth inhibitor include those illustrated below.

Local anesthetics: lidocaine, lidocaine hydrochloride, dibucaine, dibucaine hydrochloride, ethyl aminobenzoate, eucalyptus oil, eugenol, camphor, peppermint oil, and the like.

Antimicrobials: isopropylmethylphenol, chlorhexidine gluconate, chlorhexidine hydrochloride, benzalkonium chloride, benzethonium chloride, cetyltrimethylammonium bromide, dequalinium chloride, triclosan, trichlorocarbanilide, and the like.

Antivirals: acyclovir, penciclovir, and the like.

Keratin softeners: isopropyl alcohol, propanol, butanol, polyethylene glycol, benzyl alcohol, phenylethyl alcohol, propylene carbonate, hexyldodecanol, allantoin, dimethylsulfoxide, dimethylacetamide, dimethylformamide, triethanolamine, diisopropyl adipate, ethyl laurylate, lanolin, fatty acid dialkylol amide, urea, sulfur, resorcin, phytic acid, lactic acid, lactates, sodium hydroxide, potassium hydroxide, and the like.

Moisturizers: 1,3-butylene glycol, propylene glycol, dipropylene glycol, glycerol, diglycerol, high molecular weight compounds such as polyethylene glycol, diglycerol-trehalose, sodium hyaluronate, heparinoids, sodium chondroitin sulfate, collagen, elastin, keratin, chitin, chitosan, etc.; amino acids such as glycine, aspartic acid, arginine, etc.; natural moisturizing factors such as sodium lactate, urea, sodium pyrrolidone carboxylate, etc.; plant extracts such as chamomile extracts, aloe extracts, aloe vera extracts, hamamelis extracts, rosemary extracts, thyme extracts, tea extracts, perilla extracts, etc.; and the like.

Astringents: citric acid, tartaric acid, lactic acid, aluminum chloride, aluminum sulfate, allantoin chlorohydroxyaluminum, allantoin dihydroxyaluminum, aluminum phenolsulfonate, zinc paraphenolsulfonate, zinc sulfate, zinc lactate, aluminum chlorohydroxide, and the like.

Antioxidants: dibutylhydroxytoluene, butylhydroxyanisole, disodium ethylenediaminetetraacetate dihydrate (hereinafter sometimes referred to as sodium edetate), sorbic acid, sodium sulfite, and the like.

Hair growth inhibitors: isoflavones, blackberry lily extracts, dokudami (Houttuynia cordata) extracts, orris root extracts, papain enzyme, and the like.

The amounts incorporated of these local anesthetics, antipruritics, antimicrobials, antivirals, keratin softeners, moisturizers, astringents, antioxidants and hair growth inhibitors are not limited otherwise as long as the effects of the present invention are not impaired, and if desired, can be suitably selected such that the upper limit of the pharmacologically acceptable range is not exceeded. Specifically, the amount is usually 0.1 to 29 wt %, preferably 0.5 to 25 wt %, particularly preferably 1 to 20 wt %, based on the total amount of the external preparation for skin.

The method for preparing the external preparation for skin of the present invention is not limited otherwise, and can be prepared by selecting various kinds of ingredients necessary for preparing usual external preparation of skin as appropriate, followed by incorporating them into the preparation in a conventional manner.

The preferred embodiments of an external preparation for skin of the present invention are illustrated below, but should not be construed to be limited thereto.

In one embodiment, an external preparation for skin of the present invention is an external preparation for skin comprising anti-inflammatory analgesics such as indomethacin, felbinac, methyl salicylate, etc., as medically effective ingredient in an amount of 0.5 to 20 wt %, a phospholipid in an amount of 1 to 4 wt %, ethanol in an amount of 55 to 65 wt % and water in an amount of 30 to 40 wt %.

In another embodiment, an external preparation for skin of the present invention is an external preparation for skin comprising antifungals such as terbinafine, sulconazole, salts thereof, etc., preferably terbinafin hydrochloride, sulconazole nitrate, etc., as medically effective ingredient in an amount of 0.5 to 5 wt %, a phospholipid in an amount of 1 to 4 wt %, ethanol in an amount of 55 to 65 wt % and water in an amount of 30 to 40 wt %.

In another embodiment, an external preparation for skin of the present invention is an external preparation for skin comprising steroids such as hydrocortisone, prednisolone, dexamethasone, ester derivatives thereof, etc., preferably hydrocortisone acetate, hydrocortisone, hydrocortisone butyrate, prednisolone, prednisolone acetate, prednisolone valerate acetate, dexamethasone, dexamethasone acetate, etc., as medically effective ingredient in an amount of 0.01 to 1 wt %, a phospholipid in an amount of 1 to 4 wt %, ethanol in an amount of 55 to 65 wt % and water in an amount of 30 to 40 wt %.

In another embodiment, an external preparation for skin of the present invention is an external preparation for skin comprising hair restorers such as ginseng extracts (essences), menthol, dipotassium glycyrrhizinate, vitamin E compounds, soybean protein hydrolysates, etc., as medically effective ingredient in an amount of 0.001 to 1 wt %, a phospholipid in an amount of 1 to 4 wt %, ethanol in an amount of 70 to 83 wt % and water in an amount of 15 to 25 wt %.

In further embodiment, an external preparation for skin of the present invention is an external preparation for skin comprising slimming agents such as caffeine, capsaicin, etc., as medically effective ingredient in an amount of 0.0001 to 5 wt %, a phospholipid in an amount of 1 to 4 wt %, ethanol in an amount of 55 to 83 wt % and water in an amount of 15 to 43 wt %.

In further embodiment, an external preparation for skin of the present invention is an external preparation for skin comprising. antipruritics such as crotamiton, diphenhydramine, a salt thereof, etc., preferably crotamiton, diphenhydramine, diphenhydramine hydrochloride, etc., as medically effective ingredient in an amount of 0.01 to 10 wt %, a phospholipid in an amount of 1 to 4 wt %, ethanol in an amount of 60 to 83 wt % and water in an amount of 15 to 25 wt %.

Further a dose or usage of the external preparation for skin of the present invention is not limited otherwise, and usually can be used, for example, by applying it to an outer surface such as skin, etc., in an appropriate amount several times per day.

The external preparation for skin of the present invention can be prepared in various dosage forms. Examples of the dosage forms include liquids (including oils, lotions, emulsions and aerosols), gels (including liquid crystals, microemulsions and liposomes), and the like, with among them, liquids (including oils, lotions and emulsions) and gels (including liquid crystals, microemulsions and liposomes) being particularly preferable.

The external preparation for skin of the present invention can be those belonging to any categories including pharmaceuticals, quasi drug or cosmetics, and thus can find in various applications. Preferred use of the external preparation for skin of the present invention includes, for example,

pharmaceuticals including a therapeutic agent for infectious skin disease such as tinea pedis, acne, etc., or an antimicrobials therefor; a therapeutic agent for dermatitis including itch and inflammation such as eczema, rash, dry pruritus, xeroderma, chilblain, miliaria, etc., or an antipruritics therefor; a disinfectants or a therapeutic agent for damages to promote a therapeutic effect or prevent a deterioration of a disease such as incised wound, chafing, shoe sore, scratch, puncture wound, burn wound, pyogenic wound, hemorrhoid, crack, chap, etc.; a therapeutic agent for labial disease such as cheilitis, angular stomatitis, chapped lips, lip sore, etc; keratin softeners for treatment of a disease such as rough finger and keratoderma of elbow, knee, heel or malleoli, etc., or dry scaly skin; anti-inflammatory analgesics; a therapeutic agent for insect bites of mosquito, tabanus spp or bee; and the like;

quasi drugs including a drug for scalp such as hair restoration (a stimulation of hair growth) or promotion of hair growth or hair increase, and the like; drugs for prophylaxis of skin dryness, chilblain, crack, chap or rash, and the like, for skin-whitening, and for suppression of hircismus (which are used for, for example, hand skin-care, rough skin-care, labial skin-care, care of hot flush after sunburn, or to condition a skin, to improve a labial's texture, to keep skin or labial healthy, to care a skin or labial with moisture, or to provide a skin or labial, and the like); and the like; or

cosmetics for moisturising or keratin softing, and the like (which are used, for example, for hand skin-care, rough skin-care, labial skin-care, care of hot flush after sunburn, to prevent or improve a wrinkle and/or skin sag, to condition a skin, to improve a labial's texture, to keep skin or labial healthy, to provide a skin or labial with moisture, or to provide a skin or labial, etc.), and the like, but are not limited thereto. Since the external preparation for skin of the present invention can increase percutaneous absorbability of a medically effective ingredient remarkably, the application thereof to the preparation particularly required for absorption of medically effective ingredient are particularly preferable, with among them a therapeutic agent for infectious skin disease such as tinea pedis, acne, etc. (antifungals, antiacnes, etc.), a therapeutic agent for dermatitis to treat itch or inflammation (antipruritics, steroids, etc.), a therapeutic agent for insect bites, anti-inflammatory analgesics, or a drug for scalp including a drug for hair care such as hair restoration (a stimulation of hair growth) and promotion of hair growth or hair increase, etc., and the like being particularly preferable.

The external preparation for skin of the present invention can as necessary contain various ingredients that are generally used in the fields of pharmaceuticals, quasi drug or cosmetics such as base materials, surfactants, thickeners, preservatives, pH adjusters, stabilizers, irritation-reducing agents, antiseptics, coloring agents, dispersing agents, perfumes, etc., within a range that does not deteriorate storage stability, viscosity, and the like, and that does not impair the effects on a promotion of the percutaneous absorption of the present invention. These ingredients can be incorporated alone or in combination with two or more kinds thereof as arbitrary.

Base materials: hydrocarbons such as paraffin, gelled hydrocarbons, ozokerite, ceresin, petrolatum, hard fats, microcrystalline waxes, etc.; fatty acids such as lauric acid, myristic acid, palmitic acid, stearic acid, behenic acid, isostearic acid, oleic acid, linoleic acid, etc.; trifatty acid glycerides such as glyceryl tri-2-ethylhexanoate (trioctanoin), etc.; polymerized silicones such as highly polymerized methylpolysiloxane, dimethylsiloxane-methyl(polyoxyethylene)siloxane-methyl(polyoxypropylene)siloxane copolymers, dimethylsiloxane-methyl(polyoxyethylene)siloxane copolymers, dimethylsiloxane-methyl(polyoxypropylene)siloxane copolymers, polyoxyethylene-methylpolysiloxane copolymers, poly(oxyethylene-oxypropylene)-methylpolysiloxane copolymers, dimethylsiloxane-methylcetyloxysiloxane copolymers, dimethylsiloxane-methylstearoxysiloxane copolymers, methylpolysiloxane esters of alkyl acrylate copolymers, crosslinked methylpolysiloxanes, crosslinked methylphenylpolysiloxanes, crosslinked polyether-modified silicones, crosslinked alkyl polyether-modified silicones, crosslinked alkyl-modified silicones, etc.; glycol acetates such as ethylene glycol monoacetate, ethylene glycol diacetate, triethylene glycol diacetate, hexylene glycol diacetate, 2-methyl-2-propene-1,1-diol diacetate, etc.; glycol esters such as triethylene glycol divalerate, 2,2,4-trimethyl-1,3-pentanediol monoisobutyrate, 2,2,4-trimethyl-1,3-pentanediol diisobutyrate, etc.; glycol acrylates such as ethylene glycol diacrylate, diethylene glycol diacrylate, propylene glycol monoacrylate, 2,2-dimethyl-trimethylene glycol diacrylate, 1,3-butylene glycol diacrylate, etc.; glycol dinitrates such as ethylene glycol dinitrate, diethylene glycol dinitrate, triethylene glycol dinitrate, propylene glycol dinitrate, etc.; 2,2′-[1,4-phenylenedioxy]diethanol; dioxanes; polyester of butylene glycol adipate; etc.; and the like.

Surfactants: sorbitan fatty acid esters such as sorbitan monoisostearate, sorbitan monolaurate, sorbitan monopalmitate, sorbitan monostearate, diglycerol sorbitan penta-2-ethylhexylate, diglycerol sorbitan tetra-2-ethylhexylate, etc.; glyceryl fatty acids such as glyceryl monostearate, glyceryl monostearate malate, etc.; polyglyceryl fatty acids such as polyglyceryl monoisostearate, polyglyceryl diisostearate, etc.; propylene glycol fatty acid esters such as propylene glycol monostearate, etc.; hydrogenated castor oil derivatives such as polyoxyethylene hydrogenated castor oil 40 (HCO-40), polyoxyethylene hydrogenated castor oil 50, polyoxyethylene hydrogenated castor oil 60, polyoxyethylene hydrogenated castor oil 80, etc.; polyoxyethylene sorbitan fatty acid esters such as polyoxyethylene (20) sorbitan monolaurate (polysorbate 20), polyoxyethylene (20) sorbitan monostearate (polysorbate 60), polyoxyethylene (20) sorbitan monooleate (polysorbate 80), etc.; polyoxyethylene glyceryl monococoate, glycerol alkyl ethers, alkyl glucosides, polyoxyethylene cetyl ethers, stearylamine, oleylamine, etc.; and the like.

Thickeners: guar gum, locust bean gum, carrageenan, xanthan gum, dextran, methylcellulose, ethylcellulose, carboxymethylcellulose, hydroxymethylcellulose, hydroxyethylcellulose, hydroxypropylcellulose, hydroxypropylmethylcellulose, sodium alginate, propylene glycol alginate esters, polyvinyl alcohols, polyvinylpyrrolidones, polyvinyl methyl ethers, carboxyvinyl polymers, acrylic acid-alkyl methacrylate copolymers, sodium polyacrylate, polyethylene glycol, bentonite, dextrin fatty acid esters, pectin, and the like.

Preservatives: benzoic acid, sodium benzoate, dehydroacetic acid, sodium dehydroacetate, isobutyl paraoxybenzoate, isopropyl paraoxybenzoate, butyl paraoxybenzoate, ethyl paraoxybenzoate, propyl paraoxybenzoate, benzyl paraoxybenzoate, methyl paraoxybenzoate, phenoxyethanol, and the like.

pH Adjusters: inorganic acids such as hydrochloric acid, sulfuric acid, phosphoric acid, polyphosphoric acid, boric acid, etc.; organic acids such as lactic acid, acetic acid, citric acid, tartaric acid, malic acid, succinic acid, sodium succinate, oxalic acid, gluconic acid, fumaric acid, propionic acid, acetic acid, aspartic acid, epsilon-aminocaproic acid, glutamic acid, aminoethylsulfonic acid, etc.; gluconolactones; ammonium acetate; inorganic bases such as sodium bicarbonate, sodium carbonate, potassium hydroxide, sodium hydroxide, calcium hydroxide, magnesium hydroxide, etc.; organic bases such as monoethanolamine, triethanolamine, diisopropanolamine, tri-isopropanolamine, lysine, and the like.

These ingredients can be used alone or in combination of two or more kinds thereof. Also the amount incorporated is not limited otherwise, as long as the effects of the present invention are not impaired, and preferably can be suitably selected such that the upper limit of the pharmacologically acceptable range is not exceeded. Specifically, the amount may be usually 0.1 to 29 wt %, preferably 0.5 to 25 wt %, particularly preferably 1 to 20 wt %, based on the total amount of the external preparation for skin.

These external preparations for skin of the present invention can be used once or as divided doses per day in a well-known or conventional manner of usage or dose, depending on the type of dosage form to be used.

Further the present invention also encompasses a method for suppressing an increase in an acid value of phospholipid in the external preparation for skin. According to the method of the present invention, a method for suppressing an increase in an acid value of phospholipid in the external preparation for skin can be achieved by coexisting etanol and water to a phospholipid. Alternatively, a method for suppressing an increase in an acid value in the external preparation for skin of the present invention can be also achieved by existing ethanol, water and one or two or more kind(s) of ingredient selected from the group consisting of glycol, glycol ether, glycerol and diglycerol to a phospholipid.

In the method of the present invention, a phospholipid used is the same as those used in an external preparation for skin described above. The each amount incorporated of phospholipid, ethanol and water is not limited otherwise, as long as the effects of the present invention are not impaired, but for a phospholipid, is usually 0.01 to 15 wt %, preferably 0.05 to 10 wt %, particularly preferably 0.1 to 8 wt %; for ethanol, is usually 55 to 83 wt %, preferably 55 to 80 wt %, more preferably 55 to 75 wt %, particularly preferably 60 to 75 wt %; for water, is usually 15 to 43 wt %, preferably 20 to 40 wt %, particularly preferably 20 to 35 wt %, based on the total amount of the external preparation for skin. Also these glycol, glycol ether, glycerol and diglycerol can be used alone or in combination of two or more kinds thereof, and the total amount of the glycol, glycol ether, glycerol and diglycerol can be 1 to 29 wt %, preferably 1 to 20 wt %, particularly preferably 1 to 10 wt %, based on the total amount of the external preparation for skin, but is not limited otherwise, as long as the effects of the present invention are not impaired.

Further the present invention encompasses a method for improving percutaneous absorption of a medically effective ingredient in the external preparation for skin. According to the method of the present invention, an improvement of percutaneous absorbability of a medically effective ingredient in the external preparation for skin can be achieved by coexisting phospholipid, ethanol and water to the medically effective ingredient. Alternatively, an improvement in percutaneous absorbability of a medically effective ingredient in the external preparation for skin of the present invention can be achieved by coexisting phospholipid, ethanol and water as well as one or two kind(s) of ingredient(s) selected from the group consisting of glycol, glycol ether, glycerol and diglycerol to the medically effective ingredient(s).

### Examples

The following Examples describe the present invention in more detail, but are not intended to limit the scope of the present invention. In the Examples, the amounts incorporated are expressed in wt % unless otherwise indicated.

### Test Example 1

### Test for Evaluating an Acid Value

An effect of an increase in an acid value of phospholipid on external preparation for skin of the present invention was tested.

Each preparation (external preparation for skin) was prepared according to the formulation shown in the Table 1. An acid value of each these preparations was measured at (1) immediately after preparation, (2) after storing in an incubator for two weeks at 50° C. (10 mL portion of each sample was filled in brown screw tube that was shielded with aluminum foil) and (3) after ultraviolet irradiation (hereinafter abbreviated as UV irradiation) for 72 hours (10 mL portion of each sample was filled in clear ampoule tube) by the Standard methods of analysis for hygienic chemists: with Commentary 2000, 2.1.4.3, Test for change in quality, 3) Test for an acid value. The measurement of each acid value was expressed as mg of potassium hydroxide required to neutralize fatty acid that is contained in 1 g of soybean phospholipid. UV irradiation was carried out by using Photostability Testing Device (“Light-Tron LT-120 D3CJ type”, NAGANO SCIENCE CO. LTD.) equipped with D65 ramp as light source at 25° C. with 5,000 lux (hereinafter abbreviated as lx), with as the result, a testing solution being exposed to a light with an amount of cumulative irradiation of 360,000 lx/hr.

The results are shown in Table 1 below.

TABLE 1

| | | | | Comp. | Comp. | Comp. | Comp. |
|---|---|---|---|---|---|---|---|
| g/100 g | Ex. 1 | Ex. 2 | Ex. 3 | Ex. 1 | Ex. 2 | Ex. 3 | Ex. 4 |
| soybean | 2 | 2 | 2 | 2 | 2 | 2 | 2 |
| phospho- | | | | | | | |
| lipid*1 | | | | | | | |
| ethanol | 83 | 75 | 55 | 45 | 35 | 25 | — |
| purified | 15 | 23 | 43 | 53 | 63 | 73 | 98 |
| water | | | | | | | |
| immediately | 0.38 | 0.37 | 0.40 | 0.38 | 0.39 | 0.42 | 0.46 |
| after | | | | | | | |
| preparation | | | | | | | |
| after two | 0.39 | 0.40 | 0.45 | 0.53 | 0.67 | 1.01 | 1.82 |
| weeks | | | | | | | |
| at 50° C. | | | | | | | |
| 72 hours | — | 0.39 | 0.40 | — | — | 0.50 | 0.77 |
| after UV | | | | | | | |
| irradiation | | | | | | | |

*1SLP-PC70 (iodine value 90~105: product of Tsuji Oil Mill Co., Ltd.

As the results, for 50° C., the measurements of acid value of comparative examples 1 to 4 were increased by 0.15 to 1.36 from those immediately after preparation, while any those of examples 1 to 3 was found an increase of 0.05 or less, which implied to be almost nothing of increase of an acid value. Similarly, for UV irradiation, the measurements of acid value of comparative examples 3 and 4 were increased, while those of examples 2 and 3 were not increased almostly.

### Test Example 2

### Test for Evaluating an Acid Value

The effect of further addition of glycol, glycol ether, glycerol or diglycerol to external preparation for skin on an increase in an acid value of phospholipid was carried out.

Each preparation (external preparation for skin) was prepared according to the formulation shown in Table 2 below. An acid value of each these preparations was measured in a similar manner to the Test Example 1, (1) immediately after preparation, and (2) after storing in an incubator for two weeks at 50° C. (10 mL portion of each sample was filled in brown screw tube that was shielded with aluminum foil) by the Standard methods of analysis for hygienic chemists: with Commentary 2000, 2.1.4.3, Test for change in quality, 3) Test for an acid value. The measurement of each acid value was expressed as mg of potassium hydroxide required to neutralize fatty acid that is contained in 1 g of soybean phospholipid.

The results are shown in Table 2 below.

TABLE 2

| | | | | | Comp. | Comp. |
|---|---|---|---|---|---|---|
| g/100 g | Ex. 4 | Ex. 5 | Ex. 6 | Ex. 7 | Ex. 5 | Ex. 6 |
| ethanol | 55 | 65 | 75 | 83 | 45 | 85 |
| soybean | 4 | 0 | 0 | 0 | 5 | 0 |
| phospholipid*1 | | | | | | |
| soybean | 0 | 2 | 0 | 0 | 0 | 6 |
| phospholipid*2 | | | | | | |
| soybean | 0 | 0 | 1 | 0.5 | 0 | 0 |
| phospholipid*3 | | | | | | |
| purified water | 26 | 23 | 21 | 15.5 | 45 | 8 |
| propylene | 15 | 0 | 0 | 0 | 5 | 0 |
| glycol | | | | | | |
| diethylene | 0 | 10 | 0 | 0 | 0 | 0 |
| glycol | | | | | | |
| monoethyl | | | | | | |
| ether | | | | | | |
| glycerol | 0 | 0 | 3 | 0 | 0 | 1 |
| diglycerol | 0 | 0 | 0 | 1 | 0 | 0 |
| immediately | 0.807 | 0.873 | 0.241 | 0.180 | 1.00 | 1.32 |
| after | | | | | | |
| preparation | | | | | | |
| after two weeks | 0.916 | 0.966 | 0.270 | 0.189 | 1.395 | 1.75 |
| at 50° C. | | | | | | |
| ratio: after two | 1.135 | 1.106 | 1.120 | 1.050 | 1.395 | 1.325 |
| weeks at | | | | | | |
| 50° C./ | | | | | | |
| immediately | | | | | | |
| after | | | | | | |
| preparation | | | | | | |

*1SLP-PC70 (iodine value 90~105: product of Tsuji Oil Mill Co., Ltd.)
*2SLP-PC35 (iodine value 90~110: product of Tsuji Oil Mill Co., Ltd.)
*3SLP-PC55 (iodine value 85~100: product of Tsuji Oil Mill Co., Ltd.)

As the result, for 50° C., the measurements of acid value of comparative examples 5 and 6 were increased by 0.395 to 0.43 from those immediately after preparation, while any those of examples 4 to 7 was found an increase of 0.109 or less, which implied to be almost nothing of increase of an acid value. Thus the ratio of those after two weeks at 50° C./those immediately after preparation was increased by 1.325 or more for the comparative examples 5 and 6, while for the examples 4 to 7 by 1.1.135 or less.

Thus for examples 4 to 7 using various kinds of soybean phospholipids, as well as glycol, glycol ether, glycerol or diglycerol, an increase of an acid value was suppressed with a larger extent as compared to the comparative examples 5 and 6.

Therefore the external preparation for skin of the present invention was very useful since it found that it could suppress mostly an increase in an acid value of phospholipid in each example, which thus it could stabilize the preparation.

### Test Example 3

### Percutaneous Absorbability Test

The effect of percutaneous absorbability of a medically effective ingredient in an external preparation for skin of the present invention was tested.

According to a formulation shown in Table 3 below, phospholipid, sulconazole nitrate, diphenhydramine hydrochloride, dibucaine hydrochloride, menthol and camphor were dissolved in ethanol, propylene glycol (for comparative example 8, ethoxydiglycol were further contained), and the resulting mixture were mixed with purified water that had been heated separately, to prepare the desired preparation (external preparation for skin). Then 10 mL of 30% aqueous ethanol solution was added to the reservoir compartment of a vertical Franz cell, and then full-thickness skin from a hairless mouse (HR-1 strain, 7 weeks-old, male), from which the fat had been removed, was fixed between the cells, thereafter to the donor compartment was added 1 mL of the testing preparation. Twenty four hours after adding the testing preparation, the sampling was performed from the reservoir compartment and immediately thereafter, sulconazole nitrate was determined quantitatively by HPLC.

The results are shown in Table 3 below.

TABLE 3

| | | Comp. | Comp. |
|---|---|---|---|
| g/100 g | Ex. 8 | Ex. 7 | Ex. 8 |
| soybean phospholipid*1 | 1 | — | — |
| ethanol | 62 | 63 | 33 |
| purified water | 30 | 30 | 30 |
| sulconazole nitrate | 1 | 1 | 1 |
| ethoxydiglycol | — | — | 30 |
| diphenhydramine | 1 | 1 | 1 |
| hydrochloride | | | |
| dibucaine hydrochloride | 0.5 | 0.5 | 0.5 |
| l-menthol | 1 | 1 | 1 |
| dl-camphor | 0.3 | 0.3 | 0.3 |
| propylene glycol | Balance | Balance | Balance |
| permeated amount | 2089 | 913 | 87 |
| (24 hours) μg/cm² | | | |

*1SLP-PC70 (iodine value 90~105: product of Tsuji Oil Mill Co., Ltd.)

From the results of the comparative examples 7 and 8, the permeated amount of sulconazole nitrate obtained using a high content of ethanol was 10 times or more that obtained using ethoxydiglycol, which was known to promote percutaneous absorption, from which thus it could be found that a higher concentration of ethanol was preferable. Further, the permeated amount obtained in example 8 was twice or more than that obtained in the comparative example 7, from which thus it could be found that percutaneous absorbability was improved remarkably larger than that obtained when soybean phospholipid contained.

Thus, the external preparation for skin of the present invention was particularly useful since the external preparation for skin of the example 8 was the external preparation for skin with excellent percutaneous absorbability, which thus could be fully expected to exhibit a pharmacologically advantageous effect of medically effective ingredient.

### Test Example 4

### Percutaneous Absorption Test

The effect of percutaneous absorbability of a medically effective ingredient in an external preparation for skin of the present invention was tested in a similar manner to the test example 3.

Percutaneous absorbability of each testing preparation (external preparation for skin) that was prepared according to the formulation shown in Tables 4 to 9 below was determined. Then 10 mL of each reservoir solution was added to the reservoir compartment of a vertical Franz cell, and then full-thickness skin from a hairless mouse (HR-1 strain, 7-week-old, male), from which the fat had been removed, was fixed between the cells, thereafter to the donor compartment was added 1 mL of the testing preparation. Twenty-four hours after adding the testing preparation, the sampling was performed from the reservoir compartment and immediately thereafter, terbinafin hydrochloride, diphenhydramine, felbinac, 1-menthol, prednisolone valerate acetate or crotamiton was determined quantitatively by HPLC.

The results are shown in Tables 4 to 9 below.

### Antifungals:

TABLE 4

| | | Ex. | Comp. | Comp. |
|---|---|---|---|---|
| g/100 g | Ex. 9 | 10 | Ex. 9 | Ex. 10 |
| ethanol | 81 | 61 | 91 | 41 |
| purified water | 16 | 35 | 5 | 56 |
| soybean phospholipid*1 | 0 | 3 | 0 | 0 |
| soybean phospholipid*3 | 0 | 0 | 3 | 0 |
| soybean phospholipid*2 | 2 | 0 | 0 | 2 |
| terbinafin hydrochloride | 1 | 1 | 1 | 1 |
| permeated amount | 553.4 | 926.2 | 282.5 | 504.5 |
| (24 hours) μg/cm² | | | | |

reservoir solution: ethanol (30 wt %), purified water (65 wt %), HCO-50 (5 wt %)
*1SLP-PC70 (iodine value 90~105: product of Tsuji Oil Mill Co., Ltd.)
*2SLP-PC35 (iodine value 90~110: product of Tsuji Oil Mill Co., Ltd.)
*3SLP-PC55 (iodine value 85~100: product of Tsuji Oil Mill Co., Ltd.)

### Antipruritic:

TABLE 5

| | Ex. | Ex. | Comp. |
|---|---|---|---|
| g/100 g | 11 | 12 | Ex. 11 |
| ethanol | 81 | 61 | 91 |
| purified water | 16 | 35 | 5 |
| soybean phospholipid*1 | 0 | 3 | 0 |
| soybean phospholipid*3 | 0 | 0 | 3 |
| soybean phospholipid*2 | 2 | 0 | 0 |
| diphenhydramine | 1 | 1 | 1 |
| permeated amount | 6442 | 6567 | 3786 |
| (24 hours) μg/cm² | | | |

reservoir solution: ethanol (30 wt %), purified water (65 wt %), HCO-50 (5 wt %)
*1SLP-PC70 (iodine value 90~105: product of Tsuji Oil Mill Co., Ltd.)
*2SLP-PC35 (iodine value 90~110: product of Tsuji Oil Mill Co., Ltd.)
*3SLP-PC55 (iodine value 85~100: product of Tsuji Oil Mill Co., Ltd.)

### Antipruritics:

TABLE 6

| | Ex. | Ex. | Comp. |
|---|---|---|---|
| g/100 g | 13 | 14 | Ex. 12 |
| ethanol | 68.15 | 53.15 | 88.15 |
| purified water | 28.85 | 41.85 | 8.85 |
| soybean phospholipid*1 | 1 | 0 | 0 |
| soybean phospholipid*3 | 0 | 0 | 0 |
| soybean phospholipid*2 | 0 | 3 | 1 |
| crotamiton | 2 | 2 | 2 |
| permeated amount | 9260 | 15600 | 4557 |
| (24 hours) μg/cm² | | | |

reservoir solution: ethanol (30 wt %), purified water (40 wt %), PEG-400 (30 wt %)
*1SLP-PC70 (iodine value 90~105: product of Tsuji Oil Mill Co., Ltd.)
*2SLP-PC35 (iodine value 90~110: product of Tsuji Oil Mill Co., Ltd.)
*3SLP-PC55 (iodine value 85~100: product of Tsuji Oil Mill Co., Ltd.)

Anti-inflammatory analgesics:

TABLE 7

| | Ex. | Ex. | Comp. |
|---|---|---|---|
| g/100 g | 15 | 16 | Ex. 13 |
| ethanol | 71 | 56 | 91 |
| purified water | 22 | 39 | 3 |
| soybean phospholipid*1 | 0 | 1 | 2 |
| soybean phospholipid*3 | 3 | 0 | 0 |
| soybean phospholipid*2 | 0 | 0 | 0 |
| felbinac | 3 | 3 | 3 |
| diisopropanolamine | 1 | 1 | 1 |
| permeated amount | 16084 | 21276 | 11839 |
| (24 hours) μg/cm² | | | |

reservoir solution: ethanol (30 wt %), purified water (64 wt %), diisopropanolamine (1 wt %), HCO-50 (5 wt %)
*1SLP-PC70 (iodine value 90~105: product of Tsuji Oil Mill Co., Ltd.)
*2SLP-PC35 (iodine value 90~110: product of Tsuji Oil Mill Co., Ltd.)
*3SLP-PC55 (iodine value 85~100: product of Tsuji Oil Mill Co., Ltd.)

Hair restorers:

TABLE 8

| | Ex. | Ex. | Comp. |
|---|---|---|---|
| g/100 g | 17 | 18 | Ex. 14 |
| ethanol | 73 | 58 | 93 |
| purified water | 22 | 39 | 3 |
| soybean phospholipid*1 | 0 | 1 | 2 |
| soybean phospholipid*3 | 3 | 0 | 0 |
| soybean phospholipid*2 | 0 | 0 | 0 |
| diisopropanolamine | 1 | 1 | 1 |
| l-menthol | 1 | 1 | 1 |
| permeated amount | 4966 | 7100 | 3322 |
| (24 hours) μg/cm² | | | |

reservoir solution: ethanol (30 wt %), purified water (64 wt %), diisopropanolamine (1 wt %), HCO-50 (5 wt %)
*1SLP-PC70 (iodine value 90~105: product of Tsuji Oil Mill Co., Ltd.)
*2SLP-PC35 (iodine value 90~110: product of Tsuji Oil Mill Co., Ltd.)
*3SLP-PC55 (iodine value 85~100: product of Tsuji Oil Mill Co., Ltd.)

### Steroids:

TABLE 9

| | Ex. | Comp. | Comp. |
|---|---|---|---|
| g/100 g | 19 | Ex. 15 | Ex. 16 |
| ethanol | 55 | 90 | 30 |
| purified water | 41.85 | 8.85 | 66.85 |
| soybean phospholipid*1 | 0 | 0 | 3 |
| soybean phospholipid*3 | 0 | 0 | 0 |
| soybean phospholipid*2 | 3 | 1 | 0 |
| prednisolone valerate | 0.15 | 0.15 | 0.15 |
| acetate | | | |
| permeated amount | 908.0 | 64.14 | 381.0 |
| (24 hours) μg/cm² | | | |

reservoir solution: ethanol (30 wt %), purified water (40 wt %), PEG-400 (30 wt %),
*1SLP-PC70 (iodine value 90~105: product of Tsuji Oil Mill Co., Ltd.)
*2SLP-PC35 (iodine value 90~110: product of Tsuji Oil Mill Co., Ltd.)
*3SLP-PC55 (iodine value 85~100: product of Tsuji Oil Mill Co., Ltd.)

Compared the results obtained in the examples with that obtained in the comparative examples, any the external preparation for skin of the present invention (the examples 9 to 19) exhibited higher percutaneous absorbability than that obtained in the comparative example.

Therefore, the external preparation for skin of the present invention was particularly useful, since it could promote the percutaneous absorbability of a medically effective ingredient excellently, which thus could be fully expected to exhibit a pharmacologically advantageous effect of medically effective ingredient, and also using external preparation for skin of the present invention could suppress an increase in an acid value of phospholipid, which thus could be fully expected to improve in a preparation stability.

Specific preparation examples are shown below, but are not limited thereto. In the preparation examples, the amount incorporated is expressed in wt % unless otherwise indicated.

### Preparation Example 1

### Anti-Inflammatory Analgesics (Liquid)

TABLE 10

| | |
|---|---|
| soybean phospholipid | 2.0 |
| (product of Tsuji Oil Mill Co., Ltd.: SLP-PC35, | |
| iodine value: 90 to 110) | |
| absolute ethanol | 55.0 |
| purified water | 33.9 |
| indomethacin | 1.0 |
| benzyl alcohol | 4.0 |
| l-menthol | 3.0 |
| diisopropanolamine | 0.5 |
| citric acid | 0.5 |
| sodium thiosulfate | 0.1 |
| total: | 100% |

### Preparation Example 2

### Anti-Inflammatory Analgesics (Liquid)

TABLE 11

| | |
|---|---|
| soybean phospholipid | 4 |
| (product of Tsuji Oil Mill Co., Ltd.: SLP-PC70, | |
| iodine value: 90 to 105) | |
| absolute ethanol | 65 |
| purified water | 23 |
| felbinac | 3 |
| l-menthol | 3 |
| diisopropanolamine | 2 |
| total: | 100% |

### Preparation Example 3

### Antifungals (Liquid)

TABLE 12

| | |
|---|---|
| soybean phospholipid | 1.0 |
| (product of Tsuji Oil Mill Co., Ltd.: SLP-PC70, | |
| iodine value: 90 to 105) | |
| absolute ethanol | 55.0 |
| purified water | 16.59 |
| sulconazole nitrate | 1.0 |
| diphenhydramine hydrochloride | 1.0 |
| dibucaine hydrochloride | 0.5 |
| dipropylene glycol | 20.0 |
| propylene glycol | 3.0 |
| l-menthol | 1.0 |
| diisopropanolamine | 0.4 |
| dl-camphor | 0.3 |
| citric acid | 0.2 |
| dibutylhydroxytoluene | 0.01 |
| total: | 100% |

### Preparation Example 4

### Antifungals (Liquid)

TABLE 13

| | |
|---|---|
| soybean phospholipid | 1.0 |
| (product of Tsuji Oil Mill Co., Ltd.: SLP-PC55, | |
| iodine value: 85 to 100) | |
| absolute ethanol | 80.0 |
| purified water | 15.0 |
| terbinafin hydrochloride | 1.0 |
| hydroxypropylcellulose | 2.0 |
| l-menthol | 1.0 |
| total: | 100% |

### Preparation Example 5

### Hair Restorers (Liquid)

TABLE 14

| | |
|---|---|
| soybean phospholipid | 0.5 |
| (product of Tsuji Oil Mill Co., Ltd.: SLP-PC70, | |
| iodine value: 90 to 105) | |
| absolute ethanol | 63.0 |
| purified water | 30.2 |
| ginseng extracts | 2.0 |
| dipotassium glycyrrhizinate | 0.3 |
| l-menthol | 2.0 |
| polysorbate 80 | 2.0 |
| total: | 100% |

### Preparation Example 6

### Steroids (Gel)

TABLE 15

| | |
|---|---|
| soybean phospholipid | 0.5 |
| (product of Tsuji Oil Mill Co., Ltd.: SLP-PC70, | |
| iodine value: 90 to 105) | |
| absolute ethanol | 55.0 |
| purified water | 23.1 |
| crotamiton | 5.0 |
| allantoin | 0.2 |
| prednisolone valerate acetate | 0.15 |
| propylene glycol | 10.0 |
| l-menthol | 3.5 |
| triethanolamine | 1.5 |
| carboxy vinyl polymer | 1.0 |
| sodium edetate | 0.05 |
| total: | 100% |

### Preparation Example 7

### Anti-Inflammatory Analgesics (Spray)

TABLE 16

| | |
|---|---|
| ethanol | 70 |
| N-methyl-2-pyrrolidone | 5 |
| diisopropanolamine | 1.5 |
| soybean phospholipid | 0.5 |
| (product of Tsuji Oil Mill Co., Ltd.: SLP-PC90, | |
| iodine value: 90 to 105) | |
| l-menthol | 6 |
| ibuprofen | 5 |
| purified water | 12 |
| total: | 100% |

Anti-inflammatory analgesics of Preparation example 7 can be used as a mist with pump container or by spraying it with a propellant such as dimethylether or LPG.

### Preparation Example 8

### Anti-Inflammatory Analgesics (Lotion)

TABLE 17

| | |
|---|---|
| ethanol | 55 |
| sorbitan stearate | 2 |
| polysorbate 60 | 4 |
| lanolin | 3 |
| liquid paraffin | 5 |
| carboxy vinyl polymer | 0.5 |
| soybean phospholipid | 3 |
| (product of Tsuji Oil Mill Co., Ltd.: SLP-PC70, | |
| iodine value: 90 to 105) | |
| l-menthol | 6 |
| methyl salicylate | 12 |
| purified water | 9.5 |
| total: | 100% |

### Preparation Example 9

### Slimming Agents (Lotion)

TABLE 18

| | |
|---|---|
| ethanol | 65 |
| soybean phospholipid | 1 |
| (product of Tsuji Oil Mill Co., Ltd.: SLP-PC55, | |
| iodine value: 85 to 100) | |
| capsaicin | 1 |
| purified water | 30.5 |
| carboxylic polymer | 0.15 |
| caffeine | 2 |
| triethanolamine | 0.35 |
| total: | 100% |

### Preparation Example 10

### Steroids (Lotion)

TABLE 19

| | |
|---|---|
| ethanol | 60 |
| soybean phospholipid | 2 |
| (product of Tsuji Oil Mill Co., Ltd.: SLP-PC35, | |
| iodine value: 90 to 110) | |
| hydrocortisone acetate | 3 |
| diisopropanolamine | 1.5 |
| purified water | 33 |
| hydrophobized- | 0.5 |
| hydroxypropylmethylcellulose | |
| total: | 100% |

### INDUSTRIAL APPLICABILITY

The external preparation for skin of the present invention can suppress an increase in an acid value of phospholipid, which thus can be expected to improve in preparation stability of the external preparation for skin, and also the external preparation for skin of the present invention comprising the external preparation for skin and the medically effective ingredient(s) can improve in the percutaneous absorbability of a medically effective ingredient(s) remarkably, which thus can be expected to permeate the medically effective ingredient(s) through a skin efficiently, which therefore are very useful in industrial.

1. An external preparation for skin comprising a phospholipid having an iodine value of 80 to 110, ethanol in an amount of 55 to 83 wt % and water in an amount of 15 to 43 wt %.

2. The external preparation for skin according to claim 1, further comprising one or two or more kind(s) of ingredient(s) selected from the group consisting of glycol, glycol ether, glycerol and diglycerol in an amount of 5 to 29 wt %.

3. The external preparation for skin according to either claim 1 or claim 2, further comprising medically effective ingredient(s).

4. The external preparation for skin according to claim 3, wherein the medically effective ingredient(s) is one or two or more kind(s) of substance(s) selected from the group consisting of vitamin A compounds, vitamin C compounds, skin-whitening agents, anti-wrinkle agents, anti-inflammatory analgesics, antifungals, steroids, hair restorers, slimming agents and antipruritics.

5. The external preparation for skin according to claim 3, wherein the medically effective ingredient(s) is one or two or more kind(s) of substance(s) selected from the group consisting of anti-inflammatory analgesics, antifungals, steroids, hair restorers and antipruritics.

6. A method for suppressing an increase in an acid value of phospholipid characterized by coexistence of ethanol in an amount of 55 to 83 wt % and water in an amount of 15 to 43 wt % with a phospholipid having an iodine value of 80 to 110.

7. The method according to claim 6, characterized by further coexistence of one or two or more kind(s) of ingredient(s) selected from the group consisting of glycol, glycol ether, glycerol and diglycerol in an amount of 5 to 29 wt %.

8. A method for improving percutaneous absorbability of medically effective ingredient(s) in an external preparation for skin, characterized by coexistence of a phospholipid having an iodine value of 80 to 110, ethanol in an amount of 55 to 83 wt % and water in an amount of 15 to 43 wt % with the medically effective ingredient(s).

9. The method according to claim 8, characterized by further coexistence of one or two or more kind(s) of ingredient(s) selected from the group consisting of glycol, glycol ether, glycerol and diglycerol in an amount of 5 to 29 wt %.
